(19) United States
(12) Patent Application Publication
(10) Pub. No.: US 2022/0340865 A1
(43) Pub. Date: Oct. 27, 2022

(54) METHOD FOR PREPARING A FERMENTATION MEDIUM

(71) Applicant: BASF SE, Ludwigshafen (DE)

(72) Inventors: Thomas Kaeding, Lampertheim (DE); Kerstin Hage, Nienburg (DE); Rodolfo Ham-Zhu, San Diego, CA (US)

(21) Appl. No.: 17/620,786

(22) PCT Filed: Jun. 29, 2020
(86) PCT No.: PCT/EP2020/068199

§ 371 Date: Dec. 20, 2021

(30) Foreign Application Priority Data
Jul. 2, 2019 (EP) 19183954.7

(51) Int. Cl. C12N 1/20

(52) U.S. Cl. CPC C12N 1/20

(57) ABSTRACT

The present invention is directed to a method of preparing a fermentation medium comprising the steps of a) mixing components of a fermentation medium comprising a complex nutrient source in water, wherein the complex nutrient source is in an amount of 0.5-30% w/v of the fermentation medium; b) adjusting the pH of the fermentation medium obtained in step a) to a pH between pH 3.0 and pH 6.7; c) subjecting the fermentation medium subsequently to a heat treatment and thereafter adjusting the pH to a pH value suitable for cell cultivation. The present invention is further directed to a method a fermentation process comprising cultivation of the cells in the fermentation medium of the invention and the production of a compound of interest. The present invention is further directed to a method for reducing color formation and viscosity during fermentation medium preparation and during the fermentation process.

### FIELD OF THE INVENTION

The present invention relates to methods for preparing a fermentation medium and the use of such fermentation medium in an industrial fermentation process.

### BACKGROUND OF THE INVENTION

Microorganisms are widely applied as industrial workhorses for the production of valuable compounds. The biotechnological production of these compounds is conducted via fermentation and subsequent purification of the product. Many industrially relevant products are secreted to the fermentation broth in significant amounts. This allows a simple product purification process suitable for industrial application.

Industrial fermentation is typically performed in large fermenters (working volume greater than 1 m³) under aerobic conditions by controlling several process variables, including but not limited to aeration rate, stirring speed, pH, initial concentrations of various nutrients, and feeding rate profiles of one or more nutrients. To grow and produce products of interest, the microorganisms require several macronutrients, e.g., carbon, nitrogen, phosphor, sulfur, in addition to micronutrients, such as trace elements, e.g., iron, copper, zinc, etc., and vitamins. These nutrients can be provided in the fermentation medium or supplemented throughout the fermentation process via one or more feeding solutions.

Fermentation processes that are relevant for industrial application involve supplementation of large amounts of carbon source to the cells to ensure the availability of sufficient amounts of growth and availability of precursors for the product of interest. In most cases, the amount of supplied carbon source exceeds 200 g of the pure component (e.g., glucose) per initial volume of the fermentation process.

Often, industrial fermentation processes are performed with fermentation media comprising complex raw materials as nutrient sources. Common complex raw materials are for instance soybean meal, soybean hydrolysate, yeast extract, and corn steep liquor. The complex raw materials contain a mixture of proteins, carbohydrates, lipids, vitamins, minerals and other biologically relevant molecules. Using complex raw materials in fermentation processes has several advantages with respect to availability, and simultaneous provision of nutrients to the cells, such as trace elements and vitamins. This property of containing a diverse set of nutrients can be useful in cases the exact nutrient requirements of the microorganisms are unknown. However, using complex raw materials also has clear disadvantages. During the preparation of the fermentation medium, in particular during heat treatment for sterilization, as well as during the fermentation process complex chemical reactions take place within the fermentation medium leading to color development, malodor, and increase in viscosity.

Color development during the preparation of the fermentation medium and in the fermentation broth during fermentation is undesired as usually the coloring substances have to be separated from the final fermentation product in a cost-intensive manner during the downstream processing in order to achieve a certain product quality.

Also an increase in viscosity of the fermentation medium during its preparation and of the fermentation broth during the fermentation process is undesired as an increase in viscosity is usually associated with a reduced oxygen saturation of the fermentation broth and thus the necessity for an increase in energy demanding stirring for aeration. This increase in stirring is in turn associated with an increase of foam formation, which usually leads to a loss of product. The latter is often compensated by the intensive use of defoamers. However, usually defoamers have to be removed during the downstream processing in order to ensure product quality. Moreover, industrial fermenters are often operated at performance limit and thus, an increase in viscosity cannot be compensated by an increase in energy for stirring. As a consequence, productivity of the process is decreasing.

Thus, there is a need for a treatment of the fermentation medium that leads to a reduced color formation and a reduction in viscosity during media preparation and during the fermentation process.

### BRIEF SUMMARY OF THE INVENTION

The present invention is directed to a method of preparing a fermentation medium comprising a heat treatment under acidic conditions. Thus, the present invention is directed to a method of preparing a fermentation medium for the cultivation of a microbial cell comprising the steps of
a. preparing a fermentation medium by mixing in water components supporting the growth of the cells, wherein the components comprise a complex nutrient source in an amount of 0.5-30% w/v of the fermentation medium;
b. adjusting the pH of the fermentation medium obtained in step a) to a pH between pH 3.0 and pH 6.7;
c. heating the fermentation medium obtained in step b) to a temperature above 90° C. for at least 2 min;
d. adjusting the pH of the fermentation medium obtained in step c) to a pH between pH 6.8 and pH 12.0.

Furthermore, the present invention relates to a fermentation medium obtained by the method of the present invention.

The present invention further relates to a method of cultivating a microbial cell comprising the steps of
a. inoculating the fermentation medium as described herein with a microbial cell, preferably, a bacterial cell; and
b. incubating the fermentation medium comprising the microbial cell for a time and under conditions sufficient to allow growth of the microbial cell.

Moreover, the present invention is directed to a method of producing a compound of interest comprising the steps of
a. cultivating a microbial cell in the fermentation medium as described herein for a time and under conditions suitable for the microbial cell to produce the compound of interest; and
b. optionally, purifying the compound of interest from the fermentation broth.

In addition, the present invention is directed to a method for reducing viscosity formation in the fermentation broth during a fermentation process comprising the steps of
a. inoculating a fermentation medium of as described herein with a microbial cell, preferably, a bacterial cell; and
b. incubating the fermentation medium comprising the microbial cell for a time and under conditions sufficient to allow growth of the microbial cell.

### BRIEF DESCRIPTION OF THE FIGURES

FIG. 1: L*values [−] before (black columns) and after (grey columns) temperature treatment for fermentation media with pH adjustments to pH 4.0, 5.5, 6.5 and 7.8.

FIG. 2: b* values [−] before (black columns) and after (grey columns) temperature treatment for fermentation media with pH adjustments to pH 4.0, 5.5, 6.5 and 7.8.

### DETAILED DESCRIPTION OF THE INVENTION

The present invention may be understood more readily by reference to the following detailed description of the embodiments of the invention and the examples included herein.

### Definitions

Unless otherwise noted, the terms used herein are to be understood according to conventional usage by those of ordinary skill in the relevant art.

It is to be understood that as used in the specification and in the claims, “a” or “an” can mean one or more, depending upon the context in which it is used. Thus, reference to “a cell” can mean that at least one cell can be utilized.

Throughout this application, various publications are referenced. The disclosures of all of these publications and those references cited within those publications in their entireties are hereby incorporated by reference into this application in order to more fully describe the state of the art to which this invention pertains.

The term “industrial fermentation” or “industrially relevant fermentation” refers to a fermentation process in which at least 200 g carbon source per liter of initial fermentation medium is added.

A “fermentation process” describes a sequence of activities comprising the preparation of the fermentation medium and the cultivation of cells in the fermentation medium. “Cultivation of the cells” or “growth of the cells” is not understood to be limited to an exponential growth phase but can also include the physiological state of the cells at the beginning of growth after inoculation and during a stationary phase. The fermentation process can be stopped by appropriate measures that limit or prevent the growth of the cells, for instance but not being limited to reducing the temperature of the fermentation broth.

The term “added in the fermentation process” or “added during the fermentation process” regarding the amount of a certain compound of the fermentation medium describes the total amount of the compound added during the fermentation process, i.e., including an amount of the compound in the initial fermentation medium as well as an amount added during the cultivation of the cells by means of one or more feed solutions.

The term “fermentation medium” refers to a water-based solution containing one or more chemical compounds that can support growth of cells.

The term “complex nutrient source” is used herein for nutrient sources which are composed of chemically undefined compounds, i.e., compounds that are not known by their chemical formula, preferably comprising undefined organic nitrogen- and/or undefined organic carbon-containing compounds. In contrast thereto, a “chemically defined nutrient source” (e.g., “chemically defined carbon source” or “chemically defined nitrogen source”) is understood to be used for nutrient sources which are composed of chemically defined compounds. A “chemically defined component” is a component which is known by its chemical formula.

The term “complex nitrogen source” is used herein for a nutrient source that is composed of one or more chemically undefined nitrogen containing compounds, i.e., nitrogen containing compounds that are not known by their chemical formula, preferably comprising organic nitrogen containing compounds, e.g., proteins and/or amino acids with unknown composition. Preferably, the complex nitrogen source comprises one or more chemically undefined nitrogen containing proteins and/or amino acids. Preferably, the complex nitrogen source comprises one or more chemically undefined nitrogen containing proteins.

The term “complex carbon source” is used herein for a carbon source that is composed of one or more chemically undefined carbon containing compounds, i.e., carbon containing compounds that are not known by their chemical formula, preferably comprising organic carbon containing compounds, e.g., carbohydrates with unknown composition.

It is clear for the skilled person that a complex nutrient source might be a mixture of different complex nutrient sources. Thus, a complex nitrogen source can comprise a complex carbon source and vice versa and a complex nitrogen source can be metabolized by the cells in a way that it functions as carbon source and vice versa.

The term “adjusting the pH of the fermentation medium” can mean adjusting the pH of the fermentation medium either by the addition of an acid and/or a base or can mean adjusting the pH of the fermentation medium by choosing the medium components to yield the desired pH after mixing of all medium components.

The term “fermentation broth” refers to the fermentation medium comprising the cells. Hence, the term “added to the fermentation medium during the cultivation of the cells” refers to the addition of components to the fermentation medium comprising cells, i.e., to the fermentation broth.

“Trace elements” as used herein are elements taken from the list of iron, copper, manganese, zinc, cobalt, nickel, molybdenum, selenium, and boron.

The term “titer of a protein of interest” as used herein is understood as the amount of protein of interest in g per volume of fermentation broth in liter.

The term “purification” or “purifying” refers to a process in which at least one component, e.g., a protein of interest, is separated from at least another component, e.g., a particulate matter of a fermentation broth, and transferred into a different compartment or phase, wherein the different compartments or phases do not necessarily need to be separated by a physical barrier. Examples of such different compartments are two compartments separated by a filtration membrane or cloth, i.e., filtrate and retentate; examples of such different phases are pellet and supernatant or cake and filtrate, respectively.

“Parent” sequence (e.g., “parent enzyme” or “parent protein”) is the starting sequences for introduction of changes (e.g. by introducing one or more amino acid substitutions) of the sequence resulting in “variants” of the parent sequences. Thus, the term “enzyme variant” or “sequence variant” or “protein variant” are used in reference to parent enzymes that are the origin for the respective variant enzymes. Therefore, parent enzymes include wild type enzymes and variants of wild-type enzymes which are used for development of further variants. Variant enzymes differ from parent enzymes in their amino acid sequence to a certain extent; however, variants at least maintain the enzyme properties of the respective parent enzyme. In one embodiment, enzyme properties are improved in variant enzymes when compared to the respective parent enzyme. In one embodiment, variant enzymes have at least the same enzymatic activity when compared to the respective parent enzyme or variant enzymes have increased enzymatic activity when compared to the respective parent enzyme.

Enzyme variants may be defined by their sequence identity when compared to a parent enzyme. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

After aligning the two sequences, in a second step, an identity value shall be determined from the alignment. Therefore, according to the present invention the following calculation of percent-identity applies:

%-identity=(identical residues/length of the alignment region which is showing the respective sequence of this invention over its complete length)*100. Thus sequence identity in relation to comparison of two amino acid sequences according to this embodiment is calculated by dividing the number of identical residues by the length of the alignment region which is showing the respective sequence of this invention over its complete length. This value is multiplied with 100 to give “%-identity”.

For calculating the percent identity of two DNA sequences the same applies as for the calculation of percent identity of two amino acid sequences with some specifications. For DNA sequences encoding for a protein the pairwise alignment shall be made over the complete length of the coding region from start to stop codon excluding introns. For non-protein-coding DNA sequences the pairwise alignment shall be made over the complete length of the sequence of this invention, so the complete sequence of this invention is compared to another sequence, or regions out of another sequence. Moreover, the preferred alignment program implementing the Needleman and Wunsch algorithm (J. Mol. Biol. (1979) 48, p. 443-453) is “NEEDLE” (The European Molecular Biology Open Software Suite (EMBOSS)) with the programs default parameters (gapopen=10.0, gapextend=0.5 and matrix=EDNAFULL).

The term “heterologous” (or exogenous or foreign or recombinant or non-native) polypeptide is defined herein as a polypeptide that is not native to the host cell, a polypeptide native to the host cell in which structural modifications, e.g., deletions, substitutions, and/or insertions, have been made by recombinant DNA techniques to alter the native polypeptide, or a polypeptide native to the host cell whose expression is quantitatively altered or whose expression is directed from a genomic location different from the native host cell as a result of manipulation of the DNA of the host cell by recombinant DNA techniques, e.g., a stronger promoter. Similarly, the term “heterologous” (or exogenous or foreign or recombinant or non-native) polynucleotide refers to a polynucleotide that is not native to the host cell, a polynucleotide native to the host cell in which structural modifications, e.g., deletions, substitutions, and/or insertions, have been made by recombinant DNA techniques to alter the native polynucleotide, or a polynucleotide native to the host cell whose expression is quantitatively altered as a result of manipulation of the regulatory elements of the polynucleotide by recombinant DNA techniques, e.g., a stronger promoter, or a polynucleotide native to the host cell, but integrated not within its natural genetic environment as a result of genetic manipulation by recombinant DNA techniques.

With respect to two or more polynucleotide sequences or two or more amino acid sequences, the term “heterologous” is used to characterized that the two or more polynucleotide sequences or two or more amino acid sequences are naturally not occurring in the specific combination with each other.

For the purposes of the invention, “recombinant” (or transgenic) with regard to a cell or an organism means that the cell or organism contains a heterologous polynucleotide which is introduced by man by gene technology and with regard to a polynucleotide includes all those constructions brought about by man by gene technology/recombinant DNA techniques in which either
(a) the sequence of the polynucleotide or a part thereof, or
(b) one or more genetic control sequences which are operably linked with the polynucleotide, including but not limited thereto a promoter, or
(c) both a) and b)

are not located in their wildtype genetic environment or have been modified.

The term “native” (or wildtype or endogenous) cell or organism and “native” (or wildtype or endogenous) polynucleotide or polypeptide refers to the cell or organism as found in nature and to the polynucleotide or polypeptide in question as found in a cell in its natural form and genetic environment, respectively (i.e., without there being any human intervention).

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic. The term “nucleic acid construct” is synonymous with the term “expression cassette” when the nucleic acid construct contains the control sequences required for expression of a polynucleotide.

The term “control sequence” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS, shine dalgarno sequence), 3′-UTR, and transcription start and stop sites.

The term “functional linkage” or “operably linked” with respect to regulatory elements, is to be understood as meaning the sequential arrangement of a regulatory element (including but not limited to a promoter) with a nucleic acid sequence to be expressed and, if appropriate, further regulatory elements (including but not limited to a terminator) in such a way that each of the regulatory elements can fulfil its intended function to allow, modify, facilitate or otherwise influence expression of said nucleic acid sequence.

A “promoter” or “promoter sequence” is a nucleotide sequence located upstream of a gene on the same strand as the gene that enables that gene's transcription. Promoter is followed by the transcription start site of the gene. Promoter is recognized by RNA polymerase (together with any required transcription factors), which initiates transcription. A functional fragment or functional variant of a promoter is a nucleotide sequence which is recognizable by RNA polymerase, and capable of initiating transcription.

When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG, CTG or TTG and ends with a stop codon such as TAA, TAG, or TGA. The coding sequence may be a DNA, cDNA, synthetic, or recombinant nucleotide sequence. The start codon can also be named herein as “translational start signal” or “translational start site”. The stop codon can also be named herein as “translational stop signal” or “translational stop site”.

The term “expression” or “gene expression” means the transcription of a specific gene or specific genes or specific nucleic acid construct. The term “expression” or “gene expression” in particular means the transcription of a gene or genes or genetic construct into structural RNA (e.g., rRNA, tRNA) or mRNA with or without subsequent translation of the latter into a protein. The process includes transcription of DNA and processing of the resulting mRNA product.

The term “expression vector” is defined herein as a linear or circular DNA molecule that comprises a polynucleotide that is operably linked to one or more control sequences that provides for the expression of the polynucleotide.

The term “host cell”, as used herein, includes any cell type that is susceptible to transformation, transfection, transduction, conjugation, and the like with a nucleic acid construct or expression vector.

The term “introduction” and variations thereof are defined herein as the transfer of a DNA into a host cell. The introduction of a DNA into a host cell can be accomplished by any method known in the art, including, the not limited to, transformation, transfection, transduction, conjugation, and the like.

The term “donor cell” is defined herein as a cell that is the source of DNA introduced by any means to another cell.

The term “recipient cell” is defined herein as a cell into which DNA is introduced.

### DETAILED DESCRIPTION

Fermentation Medium and Method for Preparation

The present invention is directed to a method of preparing a fermentation medium comprising a heat treatment under acidic conditions. Thus, in one embodiment, the present invention is directed to a method of preparing a fermentation medium for the cultivation of a microbial cell comprising the steps of
a. preparing a fermentation medium by mixing in water components supporting the growth of the cells, wherein the components comprise a complex nutrient source in an amount of 0.5-30% w/v of the fermentation medium;
b. adjusting the pH of the fermentation medium obtained in step a) to a pH between pH 3.0 and pH 6.7;
c. heating the fermentation medium obtained in step b) to a temperature above 90° C. for at least 2 min; and
d. adjusting the pH of the fermentation medium obtained in step c) to a pH between pH 6.8 and pH 12.0.

In one embodiment the fermentation medium of the present invention comprises the medium components required for the growth of a cultivated cell. In one embodiment, the fermentation medium comprises one or more components selected from the group consisting of nitrogen source, phosphor source, sulfur source and salt, and optionally one or more further components selected the group consisting of micronutrients, like vitamins, amino acids, minerals, and trace elements. In one embodiment, the fermentation medium also comprises a carbon source. Such components are generally well known in the art (see, e.g., Ausubel, et al, Short Protocols in Molecular Biology, 3rd ed., Wiley & Sons, 1995; Sambrook, et al., Molecular Cloning: A Laboratory Manual, Second Edition, 1989 Cold Spring Harbor, N.Y.; Talbot, Molecular and Cellular Biology of Filamentous Fungi: A Practical Approach, Oxford University Press, 2001; Kinghom and Turner, Applied Molecular Genetics of Filamentous Fungi, Cambridge University Press, 1992; and Bacillus (Biotechnology Handbooks) by Colin R. Harwood, Plenum Press, 1989).

In one embodiment, the complex nutrient source is a complex nitrogen source. Complex sources of nitrogen include, but are not limited thereto protein-containing substances, such as an extract from microbial, animal or plant cells, e.g., plant protein preparations, soy meal, corn meal, pea meal, corn gluten, cotton meal, peanut meal, potato meal, meat and casein, gelatins, whey, fish meal, yeast protein, yeast extract, tryptone, peptone, bacto-tryptone, bacto-peptone, wastes from the processing of microbial cells, plants, meat or animal bodies, and combinations thereof. In one embodiment, the complex nitrogen source is selected from the group consisting of plant protein, preferably potato protein, soy protein, corn protein, peanut, cotton protein, and/or pea protein, casein, tryptone, peptone and yeast extract and combinations thereof. Preferably, the complex nutrient source is complex plant protein. Preferably, the complex nutrient source comprising one or more chemically undefined compounds is complex plant protein. Preferably, the complex nitrogen source comprising one or more chemically undefined nitrogen containing proteins is complex plant protein.

In one embodiment, the fermentation medium also comprises defined media components in addition to the one or more complex nutrient source. In one embodiment, the fermentation medium also comprises a defined nitrogen source. Examples of inorganic nitrogen sources are ammonium, nitrate, and nitrit, and combinations thereof. In a preferred embodiment, the fermentation medium comprises a nitrogen source, wherein the nitrogen source is a complex or a defined nitrogen source or a combination thereof. In one embodiment, the defined nitrogen source is selected from the group consisting of ammonia, ammonium, ammonium salts, (e.g., ammonium chloride, ammonium nitrate, ammonium phosphate, ammonium sulfate, ammonium acetate), urea, nitrate, nitrate salts, nitrit, and amino acids, preferably, glutamate, and combinations thereof.

The fermentation medium of the present invention comprises prior pH adjustment 0.5-30% complex nutrient source. In one embodiment, the complex nutrient source is in an amount of 1-20% w/v of the fermentation medium. In another embodiment, the complex nutrient source is in an amount of 2-20% w/v of the fermentation medium. In a further embodiment, prior pH adjustment the complex nutrient source is in an amount of 3-15% w/v of the fermentation medium.

In one embodiment, the fermentation medium further comprises a carbon source. The carbon source is preferably a complex or a defined carbon source or a combination thereof. In one embodiment, the complex nutrient source comprises a carbohydrate source. Various sugars and sugar-containing substances are suitable sources of carbon, and the sugars may be present in different stages of polymerisation. Preferred complex carbon sources to be used in the present invention are selected from the group consisting of molasse, corn steep liquor, cane sugar, dextrin, starch, starch hydrolysate, and cellulose hydrolysate, and combinations thereof. Preferred defined carbon sources are selected from the group consisting of carbohydrates, organic acids, and alcohols, preferably, glucose, fructose, galactose, xylose, arabinose, sucrose, maltose, lactose, acetic acid, propionic acid, lactic acid, formic acid, malic acid, citric acid, fumaric acid, glycerol, inositol, mannitol and sorbitol, and combinations thereof. Preferably, the defined carbon source is provided in form of a sirup, which can comprise up to 20%, preferably, up to 10%, more preferably up to 5% impurities. In one embodiment, the carbon source is sugar beet sirup, sugar cane sirup, corn sirup, preferably, high fructose corn sirup. In another embodiment, the complex carbon source is selected from the group consisting of molasses, corn steep liquor, dextrin, and starch, or combinations thereof, and wherein the defined carbon source is selected from the group consisting of glucose, fructose, galactose, xylose, arabinose, sucrose, maltose, dextrin, lactose, or combinations thereof.

In one embodiment, the fermentation medium is a complex medium comprising complex nitrogen and complex carbon sources. In one embodiment fermentation medium is a complex medium comprising complex nitrogen and carbon sources, wherein the complex nitrogen source may be partially hydrolyzed as described in WO 2004/003216.

In one embodiment, the fermentation medium also comprises a hydrogen source, an oxygen source, a sulfur source, a phosphorus source, a magnesium source, a sodium source, a potassium source, a trace element source, and a vitamin source.

Oxygen is usually provided during the cultivation of the cells by aeration of the fermentation media by stirring or gassing. Hydrogen is usually provided due to the presence of water in the aqueous fermentation medium. However, hydrogen and oxygen are also contained within the carbon and/or nitrogen source and can be provided that way.

Magnesium can be provided to the fermentation medium by one or more magnesium salts, preferably selected from the group consisting of magnesium chloride, magnesium sulfate, magnesium nitrate, magnesium phosphate, and combinations thereof, or by magnesium hydroxide, or by combinations of one or more magnesium salts and magnesium hydroxide.

Sodium can be added to the fermentation medium by one or more sodium salts, preferably selected from the group consisting of sodium chloride, sodium nitrate, sodium sulphate, sodium phosphate, sodium hydroxide, and combinations thereof.

Calcium can be added to the fermentation medium by one or more calcium salts, preferably selected from the group consisting of calcium sulphate, calcium chloride, calcium nitrate, calcium phosphate, calcium hydroxide, and combination thereof. Preferably, at least 0.01 g of calcium is added per liter of initial fermentation medium.

Potassium can be added to the fermentation medium by one or more potassium salts, preferably selected from the group consisting of potassium chloride, potassium nitrate, potassium sulphate, potassium phosphate, potassium hydroxide, and combination thereof. Preferably, at least 0.4 g of potassium is added per liter of initial fermentation medium.

Phosphorus can be added to the fermentation medium by one or more salts comprising phosphorus, preferably selected from the group consisting of potassium phosphate, sodium phosphate, magnesium phosphate, phosphoric acid, and combinations thereof. Preferably, at least 1 g of phosphorus is added per liter of initial fermentation medium.

Sulfur can be added to the fermentation medium by one or more salts comprising sulfur, preferably selected from the group consisting of potassium sulfate, sodium sulfate, magnesium sulfate, sulfuric acid, and combinations thereof. Preferably, at least 0.15 g of sulfur is added per liter of initial fermentation medium.

One or more trace element ions can be added to the fermentation medium in form, preferably in amounts of below 10 mmol/L initial fermentation medium each. These trace element ions are selected from the group consisting of iron, copper, manganese, zinc, cobalt, nickel, molybdenum, selenium, and boron and combinations thereof. Preferably, the trace element ions iron, copper, manganese, zinc, cobalt, nickel, and molybdenum are added to the fermentation medium. Preferably, the one or more trace element ions are added to the fermentation medium in an amount selected from the group consisting of 50 μmol to 5 mmol per liter of initial medium of iron, 40 μmol to 4 mmol per liter of initial medium copper, 30 μmol to 3 mmol per liter of initial medium manganese, 20 μmol to 2 mmol per liter of initial medium zinc, 1 μmol to 100 μmol per liter of initial medium cobalt, 2 μmol to 200 μmol per liter of initial medium nickel, and 0.3 μmol to 30 μmol per liter of initial medium molybdenum, and combinations thereof. For adding each trace element preferably one or more from the group consisting of chloride, phosphate, sulphate, nitrate, citrate and acetate salts can be used.

Compounds which may optionally be included in a fermentation medium are chelating agents, such as citric acid, MGDA, NTA, or GLDA, and buffering agents such as mono- and dipotassium phosphate, calcium carbonate, and the like. Preferably, the fermentation medium comprises citric acid. Buffering agents preferably are added when dealing with processes without an external pH control. In addition, an antifoaming agent may be dosed prior to and/or during the fermentation process.

Vitamins refer to a group of structurally unrelated organic compounds which are necessary for the normal metabolism of cells. Cells are known to vary widely in their ability to synthesize the vitamins they require. A vitamin should be added to the fermentation medium of Bacillus cells not capable to synthesize said vitamin. Vitamins can be selected from the group of thiamin, riboflavin, pyridoxal, nicotinic acid or nicotinamide, pantothenic acid, cyanocobalamin, folic acid, biotin, lipoic acid, purines, pyrimidines, inositol, choline and hemins.

In on embodiment, the fermentation medium also comprises a selection agent, e.g., an antibiotic, such as ampicillin, tetracycline, kanamycin, hygromycin, bleomycin, chloroamphenicol, streptomycin or phleomycin, to which the selectable marker of the cells provides resistance.

The amount of necessary compounds to be added to the fermentation medium will mainly depend on the amount of biomass which is to be formed in the fermentation process. The amount of biomass formed may vary widely, typically from about 10 to about 150 grams of dry cell mass per liter of fermentation broth. Usually, for protein production, fermentations producing an amount of biomass which is lower than about 10 g of dry cell mass per liter of fermentation broth are not considered industrially relevant.

The optimum amount of each component of a fermentation medium, as well as which compounds are essential and which are non-essential, will depend on the type of cell which is subjected to fermentation in the medium, on the amount of biomass and on the product to be formed. Typically, the amount of medium components necessary for growth of the cell may be determined in relation to the amount of carbon source used in the fermentation, since the amount of biomass formed will be primarily determined by the amount of carbon source used.

An industrially relevant fermentation process preferably encompasses a fermentation process on a volume scale which is at least 1 m3 with regard to the nominal fermenter size, preferably at least 5 m3, more preferably at least 10 m3, even more preferably at least 25 m3, most preferably at least 50 m3.

Preferably, prior inoculation the fermentation medium and feed solutions are sterilized in order to prevent or reduce growth of unwanted microorganisms during the fermentation process. Sterilization can be performed with methods known in the art, for example but not limited to heat sterilization or sterile filtration. Medium components can be sterilized separately from other medium components to avoid interactions of medium components during sterilization treatment or to avoid decomposition of medium components under sterilization conditions.

In one embodiment, a carbohydrate source is added after step c) or after step d) to the fermentation medium.

In one embodiment, the pH of the fermentation medium before heat treatment is adjusted by adding an appropriate acid or base. Preferred acids are selected from sulfuric acid and phosphoric acid. Preferred bases are selected from ammonia and sodium hydroxide. In a preferred embodiment, the pH of the fermentation medium before heat treatment is adjusted by adding an appropriate acid. In one embodiment, the pH of the fermentation medium before heat treatment is adjusted by the addition of the last component of the fermentation medium. In one embodiment, after combining all components of the fermentation medium and before heat treatment of the fermentation medium the pH of the fermentation medium is between pH 3.0 and pH 6.7. Thus, in one embodiment the pH adjustment of step b) is achieved by combining and at least partially solubilizing all components of the fermentation medium without the need for further pH adjustment. In one embodiment, the pH is adjusted by an acidic salt solution. In a preferred embodiment, the pH is adjusted by an acidic trace element solution. In another embodiment, after combining all components of the fermentation medium and before heat treatment of the fermentation media the pH of the fermentation medium is not between pH 3.0 and pH 6.7. In this embodiment, further pH adjustment is needed with an appropriate acid or base to obtain in step b) the pH of between pH 3.0 and pH 6.7.

In the method of the present invention, the pH of the fermentation medium before heat treatment in step c) is between pH 3.0 and pH 6.7, preferably between pH 4.0 and pH 6.7, preferably between pH 4.5 and pH 6.5, more preferably between pH 5.0 and pH 6.0. The pH in step b) is set to be between pH 3.0 and pH 6.7. In another embodiment, the pH in step b) is set to be between pH 4.0 and pH 6.7. In a preferred embodiment, the pH in step b) is set to be between pH 4.5 and pH 6.5. In another a preferred embodiment, the pH in step b) is set to be between pH 5.0 and pH 6.0. As understood herein, the pH in step b) can be adjusted by selecting the appropriate medium components in step a) making it unnecessary to further adjust the pH under step b), e.g., by the addition of acid or base.

In one embodiment, the heat treatment of the fermentation medium in step c) is with saturated steam. In one embodiment, the heat treatment of the fermentation medium in step c) is at a temperature and for a time longer or shorter than needed to obtain sterilization of the fermentation medium. In another embodiment, the heat treatment of the fermentation medium in step c) is at a temperature and for a time suitable to obtain sterilization of the fermentation medium. In a further embodiment, the heat treatment of the fermentation medium in step c) is with saturated steam at a temperature equal or above 120° C. for at least 20 min. In a further embodiment, the heat treatment of the fermentation medium in step c) is with saturated steam at a temperature equal or above 140° C. for at least 2 min. In yet another embodiment, the heat treatment of the fermentation medium in step c) is with saturated steam at a temperature equal or above 120° C. for 40-60 min.

In the method of the present invention, the pH of the fermentation medium after heat treatment in step c) is adjusted between pH 6.8 and pH 12.0, preferably between pH 6.8 and pH 9, more preferably between pH 7.0 and pH 8.5, most preferably between pH 7.0 and pH 8.0. The pH after step d) is set to be between pH 6.8 and pH 12.0. In another embodiment, the pH after step d) is set to be between pH 6.8 and pH 9. In yet a preferred embodiment, the pH in step d) is set to be between pH 7.0 and pH 9.0. In a further preferred embodiment, the pH in step d) is set to be between pH 7.0 and pH 8.5. In an even more preferred embodiment, the pH in step d) is set to be between pH 7.0 and pH 8.0. As understood herein, the pH in step d) can be adjusted by selecting the appropriate medium components in step a) and/or the appropriate conditions in step c) making it unnecessary to further adjust the pH under step d), e.g., by the addition of acid or base.

In one embodiment, the present invention is directed to a fermentation medium obtained by a method described herein.

Preferably, the present invention is directed to a method of preparing a fermentation medium comprising the steps of
a. mixing components of a fermentation medium comprising a complex nutrient source in water, wherein the complex nutrient source is in an amount of 0.5-30% w/v of the fermentation medium;
b. adjusting the pH of the fermentation medium obtained in step a) to a pH between pH 5.0 and pH 6.6, preferably between pH 5.0 and pH 6.0;
c. treating the fermentation medium obtained in step b) with a temperature above 90° C. for at least 2 min, preferably, equal or equivalent to treatment with saturated steam at a temperature equal or above 120° C. for at least 20 min;
d. adjusting the pH of the fermentation medium obtained in step c) to a pH between pH 7.0 and pH 9.0.

In one embodiment, the fermentation medium comprises all components necessary to support growth of a microbial cell, preferably a bacterial cell. Components necessary to support growth of a microbial cell, preferably a bacterial cell, are described herein.

Method of Cultivation

One embodiment of the present invention is a method of cultivating a cell comprising the steps of
a) inoculating the fermentation medium obtained by the method described herein with a microbial cell, preferably, a bacterial cell, most preferably a Bacillus cell; and
b) incubating the fermentation medium comprising the cell for a time and under conditions sufficient to allow growth of the microbial cell.

Thus, part of the present invention is the use of a fermentation medium described herein for the cultivation of a microorganism.

The fermentation may be performed as a batch, a repeated batch, a fed-batch, a repeated fed-batch or a continuous fermentation process. In a fed-batch process, either none or part of the compounds comprising one or more of the structural and/or catalytic elements, like carbon or nitrogen source, is added to the medium before the start of the fermentation and either all or the remaining part, respectively, of the compounds comprising one or more of the structural and/or catalytic elements are fed during the fermentation process. The compounds which are selected for feeding can be fed together or separate from each other to the fermentation process. In a repeated fed-batch or a continuous fermentation process, the complete start medium is additionally fed during fermentation. The start medium can be fed together with or separate from the feed(s). In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth. In a preferred embodiment of the invention, a fed-batch fermentation process is preferred.

Many cell cultures incorporate a carbon source, like glucose, as a substrate feed in the cell culture during fermentation. Thus, in one embodiment, the method of cultivating the microorganism comprises a feed comprising a carbon source. The carbon source containing feed can comprise a defined or a complex carbon source as described in detail herein, or a mixture thereof. In one embodiment, a chelating agent is comprised in the carbon source feed.

The fermentation time, pH, temperature, antifoam or other specific fermentation conditions may be applied according to standard conditions known in the art. In one embodiment, the fermentation conditions are adjusted to obtain maximum yields of the protein of interest.

Preferably, the temperature of the fermentation broth during cultivation is 25° C. to 45° C., preferably, 27° C. to 40° C., more preferably, 27° C. to 37° C.

Preferably, the pH of the fermentation broth during cultivation of the Bacillus cells is adjusted to at or above pH 6.8, pH 7.0, pH 7.2, pH 7.4, or pH 7.6. Preferably, the pH of the fermentation broth during cultivation of the Bacillus cells is adjusted to pH 6.8 to 9, preferably to pH 6.8 to 8.5, more preferably to pH 7.0 to 8.5, most preferably to pH 7.2 to pH 8.0.

In one embodiment, fermentation is carried out with stirring and/or shaking the fermentation medium. The agitation rate depends on the fermenter size and the appropriate setting is within the knowledge of the skilled person.

In one embodiment, oxygen is added to the fermentation medium during cultivation, preferably by stirring and/or agitation as described herein or by gassing, preferably with 0-3 bar air or oxygen. In one embodiment, fermentation is performed under saturation with oxygen.

In one embodiment, the fermentation time is for 1-200 hours, preferably, 1-120 hours, more preferably 10-60 h, even more preferably, 20-50 h.

Culture conditions for a given cell type may also be found in the scientific literature and/or from the source of the cell such as the American Type Culture Collection (ATCC) and Fungal Genetics Stock Center.

It has been surprisingly found by the present inventors that preparing the fermentation medium as described herein leads to improved properties of the fermentation medium. These improved properties are for instance a reduced increase in viscosity of the fermentation media after heat treatment, preferably after sterilization. The use of the fermentation media obtained by the method as described herein further leads to a limitation of the increase in viscosity of the fermentation broth during the fermentation process. Thus, compared to the use of a fermentation method not obtained by the method described herein, but comprising the same media components, the viscosity of the fermentation medium after sterilization is lower and/or the viscosity increase of the fermentation broth during the fermentation process is lower.

Thus, another aspect of the present invention is a method for reducing viscosity formation in the fermentation broth during a fermentation process comprising the steps of

a) inoculating a fermentation medium obtained by a method described herein with a microbial cell, preferably, a bacterial cell; and

b) incubating the fermentation medium comprising the microbial cell for a time and under conditions sufficient to allow growth of the microbial cell.

Thus, the fermentation medium described herein can be used for limiting the increase in viscosity of the fermentation broth during a fermentation process.

Thus, another aspect of the present invention is a method for limiting the increase in viscosity of the fermentation broth during a fermentation process comprising the steps of

a) inoculating a fermentation medium obtained by a method described herein with a microbial cell, preferably, a bacterial cell; and

b) incubating the fermentation medium comprising the microbial cell for a time and under conditions sufficient to allow growth of the microbial cell.

The cells are cultivated in a fermentation medium described herein, in particular, in a fermentation medium prepared by the method described herein.

One embodiment of the present invention is a method of cultivating a cell comprising the steps of
a. mixing components of a fermentation medium comprising a complex nutrient source in water, wherein the complex nutrient source is in an amount of 0.5-30% w/v of the fermentation medium;
b. adjusting the pH of the fermentation medium obtained in step a) to a pH between pH 3.0 and pH 6.7;
c. treating the fermentation medium obtained in step b) with a temperature above 90° C. for at least 2 min;
d. adjusting the pH of the fermentation medium obtained in step c) to a pH between pH 6.8 and pH 12.0;
e. inoculating the fermentation medium obtained by the method described herein with a microbial cell, preferably, a bacterial cell, most preferably a Bacillus cell; and
f. incubating the fermentation medium comprising the cell for a time and under conditions sufficient to allow growth of the microbial cell.

Preferably, the present invention is directed to a method of preparing a fermentation medium comprising the steps of
a. mixing components of a fermentation medium comprising a complex nutrient source in water, wherein the complex nutrient source is in an amount of 0.5-30% w/v of the fermentation medium;
b. adjusting the pH of the fermentation medium obtained in step a) to a pH between pH 5.0 and pH 6.6, preferably between pH 5.0 and pH 6.0;
c. treating the fermentation medium obtained in step b) with a temperature above 90° C. for at least 2 min, preferably with standard autoclaving conditions, preferably, equal or equivalent to treatment with saturated steam at a temperature equal or above 120° C. for at least 20 min;
g. adjusting the pH of the fermentation medium obtained in step c) to a pH between pH 7.0 and pH 9.0
h. inoculating the fermentation medium obtained by the method described herein with a microbial cell, preferably a bacterial cell, most preferably a Bacillus cell; and
i. incubating the fermentation medium comprising the cell for a time and under conditions sufficient to allow growth of the microbial cell.

Thus, the fermentation medium described herein can be used for limiting the increase in viscosity of the fermentation medium after heat treatment, preferably after sterilization, prior inoculation.

It has also been surprisingly found by the present inventors that preparing the fermentation media as described herein leads to a reduction of the formation of compounds that provide an undesired color to the fermentation media. The use of the fermentation media obtained by the method as described herein also leads to a reduction of the formation of color of the fermentation broth during fermentation.

Therefore, another aspect of the present invention is a method for limiting the increase in color formation, preferably yellow and/or brown color formation, in the fermentation broth during a fermentation process comprising the steps of

a) inoculating a fermentation medium as described herein with a microbial cell, preferably, a bacterial cell; and

b) incubating the fermentation medium comprising the cell for a time and under conditions sufficient to allow growth of the microbial cell.

In a preferred embodiment, color formation is measured by determining the Lab-values using methods known in the art.

Thus, the fermentation medium described herein can be used for limiting the color formation, preferably yellow and/or brown color formation, of the fermentation broth during a fermentation process.

Therefore, another aspect of the present invention is a method for reducing the increase in color formation, preferably yellow and/or brown color formation, in the fermentation medium after heat treatment, preferably after sterilization, prior inoculation with cells comprising the steps of
a) inoculating a fermentation medium prepared by the method described herein with a microbial cell, preferably, a bacterial cell; and
b) incubating the fermentation medium comprising the cell for a time and under conditions sufficient to allow growth of the microbial cell.

Thus, the fermentation medium described herein can be used for limiting the increase color formation, preferably yellow and/or brown color formation, in the fermentation medium after heat treatment, preferably after sterilization, prior inoculation.

Cultivated Cells

The microbial cell (also called herein microorganism or microbe) for cultivation in the fermentation medium described herein can be a prokaryotic or a eukaryotic cell. In one embodiment, the microorganism is a bacteria, an archaea, a fungal cell, or a yeast cell. Preferably, the microbial cell is a bacterial cell, preferably a Bacillus cell. In one embodiment, the microbial cell comprises one or more genetic constructs for heterologous gene expression.

Useful prokaryotes are bacterial cells such as gram positive or gram negative bacteria.

Gram-positive bacteria include, but are not limited to, Bacillus, Brevibacterium, Corynebacterium, Streptococcus, Streptomyces, Staphylococcus, Enterococcus, Lactobacillus, Lactococcus, Clostridium, Geobacillus, and Oceanobacillus.

In the methods of the present invention, the bacterial cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus methylotrophicus, Bacillus cereus Bacillus paralicheniformis, Bacillus subtilis, and Bacillus thuringiensis cells. In one embodiment, the bacterial host cell is a Bacillus amyloliquefaciens, Bacillus lentus, Bacillus licheniformis, Bacillus stearothermophilus or Bacillus subtilis cell. In another embodiment, the bacterial host cell is a Bacillus licheniformis cell or a Bacillus subtilis cell, in a specific embodiment a Bacillus licheniformis cell. In one embodiment, the Bacillus cell is a Bacillus cell of Bacillus subtilis, Bacillus pumilus, Bacillus licheniformis, or Bacillus lentus. Preferably, the cell is a Bacillus licheniformis cell.

In the methods of the present invention, the bacterial cell may be Lactobacillus acidophilus, Lactobacillus plantarum, Lactobacillus gasseri, Lactobacillus bulgaricusk, Lactobacillus reuteri, Escherichia coli, Staphylococcus aureus, Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola, Corynebacterium effiziens, Corynebacterium efficiens, Corynebacterium deserti, Brevibacterium flavum, Brevibacterium lactofermentum, Brevibacterium divarecatum, Pseudomonas putida, Pseudomonas syringae, Streptomyces coelicolor, Streptomyces lividans, Streptomyces albus, Streptomyces avermitilis, Gluconobacter oxydans, Gluconobacter morbifer, Gluconobacter thailandicus, Acetobacter aceti, Clostridium acetobutylicum, Clostridium saccharobutylicum, Clostridium beijerinckii, Streptococcus equisimilis, Streptococcus pyogenes, Streptococcus uberis, Streptococcus equi subsp., Zooepidemicus or Basfia succiniciproducens.

Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

Gram-negative bacteria include, but are not limited to, Escherichia, Pseudomonas, Salmonella, Campylobacter, Helicobacter, Acetobacter, Flavobacterium, Fusobacterium, Gluconobacter. In a specific embodiment, the bacterial host cell is a Escherichia coli cell.

Another gram negative bacteria is Pseudomonas purrocinia (ATCC 15958) or Pseudomonas fluorescens (NRRL B-11), or Basfia succiniciproducens.

In a specific embodiment the microbial cell is of the genus Escherichia or Bacillus.

The microorganism may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), Myceliophthora, Thermothelomyces, preferably, Thermothelomyces thermohila, and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, e.g., Aspergillus niger, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A).

Further preferred fungi include strains belonging to the subdivision Zygomycotina, class Mycoraceae, e.g. Rhizopus or Mucor, in particular Mucor hiemalis.

The fungal cell may be a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida). In another embodiment, the fungal host cell is a filamentous fungal cell, e.g., Ashbya spec, preferably Ashbya gossypii (Eremothecium gossypii).

The nucleic acid construct comprising the gene encoding the protein of interest comprises one or more promoter sequences that directs the expression of the gene of interest in the host cell and further comprises a transcription and translation start and terminator.

In one embodiment, the nucleic acid construct and/or the expression vector comprising the gene of interest comprises one or more further control sequences. Such control sequences include, but are not limited to promoter sequence, 5′-UTR (also called leader sequence), ribosomal binding site (RBS, shine dalgarno sequence), and 3′-UTR.

The compound of interest produced by the microbial cell may be secreted (into the liquid fraction of the fermentation broth) or may remain inside the cell. For secretion of a protein of interest into the fermentation medium the nucleic acid construct comprises a polynucleotide encoding for a signal peptide that directs secretion of the protein of interest into the fermentation medium. Various signal peptides are known in the art. Preferred signal peptides are selected from the group consisting of the signal peptide of the AprE protein from Bacillus subtilis or the signal peptide from the YvcE protein from Bacillus subtilis.

In particular, suitable for secreting amylases from Bacillus cells into the fermentation medium are the signal peptide of the AprE protein from Bacillus subtilis or the signal peptide from the YvcE protein from Bacillus subtilis. As the YvcE signal peptide is suitable for secreting a wide variety of different amylases this signal peptide can be used, preferably in conjunction with the fermentation process described herein, for expressing a variety of amylases and analyzing the amylases regarding their properties, e.g., amylolytic activity or stability.

In one embodiment, the expression vector comprising the gene of interest is located outside the chromosomal DNA of the host cell. In another embodiment, the expression vector is integrated into the chromosomal DNA of the host cell. The expression vector can be linear or circular. In one embodiment, the expression vector is a viral vector or a plasmid.

For autonomous replication, the expression vector may further comprise an origin of replication enabling the vector to replicate autonomously in the host cell in question. Bacterial origins of replication include but are not limited to the origins of replication of plasmids pBR322, pUC19, pSC101, pACYC177, and pACYC184 permitting replication in E. coli (Sambrook, J. and Russell, D. W. Molecular cloning. A laboratory manual, 3rd ed, Cold Spring Harbor Laboratory Press, Cold Spring Harbor, N.Y. 2001; Cohen, S. N., Chang, A. C. Y., Boyer, H. W., & Helling, R. B. (1973). Construction of Biologically Functional Bacterial Plasmids In Vitro. Proceedings of the National Academy of Sciences of the United States of America, 70(11), 3240-3244), and pUB110, pC194, pTB19, pAMß1, and pTA1060 permitting replication in Bacillus (Janniere, L., Bruand, C., and Ehrlich, S. D. (1990). Structurally stable Bacillus subtilis cloning vectors. Gene 87, 53-6; Ehrlich, S. D., Bruand, C., Sozhamannan, S., Dabert, P., Gros, M. F., Janniere, L., and Gruss, A. (1991). Plasmid replication and structural stability in Bacillus subtilis. Res. Microbiol. 142, 869-873), and pE194 (Dempsey, L. A. and Dubnau, D. A. (1989). Localization of the replication origin of plasmid pE194. J. Bacteriol. 171, 2866-2869). The origin of replication may be one having a mutation to make its function temperature-sensitive in the host cell (see, e.g., Ehrlich, 1978, Proceedings of the National Academy of Sciences USA 75:1433-1436).

In one embodiment, the expression vector contains one or more selectable markers that permit easy selection of transformed cells. A selectable marker is a gene encoding a product, which provides for biocide resistance, resistance to heavy metals, prototrophy to auxotrophs, and the like. Bacterial selectable markers include but are not limited to the dal genes from Bacillus subtilis or Bacillus licheniformis, or markers that confer antibiotic resistance such as ampicillin, kanamycin, erythromycin, chloramphenicol or tetracycline resistance. Furthermore, selection may be accomplished by co-transformation, e.g., as described in WO91/09129, where the selectable marker is on a separate vector.

In one embodiment, the bacterial cell can be a standard E. coli or B. subtilis cloning host cell. In one embodiment, the bacterial cell can be a standard E. coli or B. subtilis cloning host cell, which has been modified by gene technology. Standard E. coli cloning hosts include but are not limited to DH5alpha (Invitrogen), DH10B, (Invitrogen), Omnimax (Invitrogen), INV110 (Invitrogen), TOP10 (Invitrogen), HB101 (Promega), SURE (Stratagene), XL1-Blue (Stratagen), TG1 (Lucigen), and JM109 (NEB). These E. coli hosts are defective in the EcoKI restrictionmodification systems, some in addition defective in the methylation-dependent restrictases mcrA, mcrB, mcrC, mrr, some in addition defective in dam and dcm DNA-methyltransferases. Bacillus subtilis cloning hosts such B. subtilis carrying a defective hsd(RI)R-M- locus such as B. subtilis IG-20 (BGSC 1A436) or a defective hsdRM1 mutation such as B. subtilis 1012 WT (Mobitec).

In another embodiment, the bacterial cell may additionally contain modifications, e.g., deletions or disruptions, of other genes that may be detrimental to the production, recovery or application of a polypeptide of interest. In one embodiment, a bacterial host cell is a protease-deficient cell. In another embodiment, the bacterial host cell comprises a disruption or deletion of one of the genes involved in the biosynthesis of polyglutamic acid.

Method of Producing a Compound of Interest

The compound of interest may or may not be further purified from the fermentation broth. Thus, in one embodiment, the present invention refers to a fermentation broth comprising a compound of interest obtained by a fermentation process as described herein.

In one embodiment, the present invention is directed to a method of producing a compound of interest comprising the steps of
a) cultivating a microbial cell in the fermentation medium described herein for a time and under conditions suitable for the microbial cell to produce the compound of interest; and
b) optionally, purifying the compound of interest from the fermentation broth.

The compound of interest can be produced by a microbial cell expressing one or more polynucleotides native or heterologous to the cells. In one embodiment, the cell is a recombinant cell created by the methods described herein.

Preferably, the compound of interest is a protein of interest. Preferably, the protein of interest is encoded by a polynucleotide heterologous to the microbial cell. The protein of interest can be produced by a cell expressing one or more polynucleotides native or heterologous to the cell coding for the protein of interest.

Preferably, the protein of interest is an enzyme. In a particular embodiment, the enzyme is classified as an oxidoreductase (EC 1), a transferase (EC 2), a hydrolase (EC 3), a lyase (EC 4), an Isomerase (EC 5), or a Ligase (EC 6) (EC-numbering according to Enzyme Nomenclature, Recommendations (1992) of the Nomenclature Committee of the International Union of Biochemistry and Molecular Biology including its supplements published 1993-1999). In a preferred embodiment, the protein of interest is a detergent enzyme.

Most preferably, the enzyme is a hydrolase (EC 3), preferably, a glycosidase (EC 3.2) or a peptidase (EC 3.4). Especially preferred enzymes are enzymes selected from the group consisting of an amylase (in particular an alpha-amylase (EC 3.2.1.1)), a cellulase (EC 3.2.1.4), a lactase (EC 3.2.1.108), a mannanase (EC 3.2.1.25), a lipase (EC 3.1.1.3), a phytase (EC 3.1.3.8), a nuclease (EC 3.1.11 to EC 3.1.31), and a protease (EC 3.4); in particular an enzyme selected from the group consisting of amylase, protease, lipase, mannanase, phytase, xylanase, phosphatase, glucoamylase, nuclease, and cellulase, preferably, amylase or protease, preferably, a protease. Most preferred is a serine protease (EC 3.4.21), preferably a subtilisin protease.

Proteases are members of class EC 3.4. Proteases include aminopeptidases (EC 3.4.11), dipeptidases (EC 3.4.13), dipeptidyl-peptidases and tripeptidyl-peptidases (EC 3.4.14), peptidyldipeptidases (EC 3.4.15), serine-type carboxypeptidases (EC 3.4.16), metallocarboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), endopeptidases of unknown catalytic mechanism (EC 3.4.99). At least one protease may be selected from serine proteases (EC 3.4.21). Serine proteases or serine peptidases (EC 3.4.21) are characterized by having a serine in the catalytically active site, which forms a covalent adduct with the substrate during the catalytic reaction. A serine protease may be selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5,) and subtilisin (also known as subtilopeptidase, e.g., EC 3.4.21.62), the latter hereinafter also being referred to as “subtilisin”. A sub-group of the serine proteases tentatively designated subtilases has been proposed by Siezen et al. (1991), Protein Eng. 4:719-737 and Siezen et al. (1997), Protein Science 6:501-523. They are defined by homology analysis of more than 170 amino acid sequences of serine proteases previously referred to as subtilisin-like proteases. A subtilisin was previously often defined as a serine protease produced by Gram-positive bacteria or fungi, and according to Siezen et al. now is a subgroup of the subtilases. A wide variety of subtilases have been identified, and the amino acid sequence of a number of subtilases has been determined. For a more detailed description of such subtilases and their amino acid sequences reference is made to Siezen et al. (1997), Protein Science 6:501-523. The subtilases may be divided into 6 subdivisions, i.e. the subtilisin family, thermitase family, the proteinase K family, the lantibiotic peptidase family, the kexin family and the pyrolysin family. A subgroup of the subtilases are the subtilisins which are serine proteases from the family S8 as defined by the MEROPS database (http://merops.sanger.ac.uk). Peptidase family S8 contains the serine endopeptidase subtilisin and its homologues.

Prominent Members of Family S8, Subfamily A are:

| name | MEROPS Family S8, Subfamily A |
|---|---|
| Subtilisin Carlsberg | S08.001 |
| Subtilisin lentus | S08.003 |
| Thermitase | S08.007 |
| Subtilisin BPN′ | S08.034 |
| Subtilisin DY | S08.037 |
| Alkaline peptidase | S08.038 |
| Subtilisin ALP 1 | S08.045 |
| Subtilisin sendai | S08.098 |
| Alkaline elastase YaB | S08.157 |

Parent proteases of the subtilisin type (EC 3.4.21.62) and variants may be bacterial proteases. Said bacterial protease may be a Gram-positive bacterial polypeptide such as a Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, or Ureaplasma protease. A review of this family is provided, for example, in “Subtilases: Subtilisin-like Proteases” by R. Siezen, pages 75-95 in “Subtilisin enzymes”, edited by R. Bott and C. Betzel, New York, 1996. At least one protease may be selected from the following: subtilisin from Bacillus amyloliquefaciens BPN′ (described by Vasantha et al. (1984) J. Bacteriol. Volume 159, p. 811-819 and J A Wells et al. (1983) in Nucleic Acids Research, Volume 11, p. 7911-7925); subtilisin from Bacillus licheniformis (subtilisin Carlsberg; disclosed in E L Smith et al. (1968) in J. Biol Chem, Volume 243, pp. 2184-2191, and Jacobs et al. (1985) in Nucl. Acids Res, Vol 13, p. 8913-8926); subtilisin PB92 (original sequence of the alkaline protease PB92 is described in EP 283075 A2); subtilisin 147 and/or 309 (Esperase®, Savinase®, respectively) as disclosed in WO 89/06279; subtilisin from Bacillus lentus as disclosed in WO 91/02792, such as from Bacillus lentus DSM 5483 or the variants of Bacillus lentus DSM 5483 as described in WO 95/23221; subtilisin from Bacillus alcalophilus (DSM 11233) disclosed in DE 10064983; subtilisin from Bacillus gibsonii (DSM 14391) as disclosed in WO 2003/054184; subtilisin from Bacillus sp. (DSM 14390) disclosed in WO 2003/056017; subtilisin from Bacillus sp. (DSM 14392) disclosed in WO 2003/055974; subtilisin from Bacillus gibsonii (DSM 14393) disclosed in WO 2003/054184; subtilisin having SEQ ID NO: 4 as described in WO 2005/063974; subtilisin having SEQ ID NO: 4 as described in WO 2005/103244; subtilisin having SEQ ID NO: 7 as described in WO 2005/103244; and subtilisin having SEQ ID NO: 2 as described in application DE 102005028295.4. At least one subtilisin may be subtilisin 309 (which might be called Savinase® herein) as disclosed as sequence a) in Table I of WO 89/06279 or a variant which is at least 80% identical thereto and has proteolytic activity. Proteases are known as comprising the variants described in: WO 92/19729, WO 95/23221, WO 96/34946, WO 98/20115, WO 98/20116, WO 99/11768, WO 01/44452, WO 02/088340, WO 03/006602, WO 2004/03186, WO 2004/041979, WO 2007/006305, WO 2011/036263, WO 2011/036264, and WO 2011/072099. Suitable examples comprise especially protease variants of subtilisin protease derived from SEQ ID NO:22 as described in EP 1921147 (with amino acid substitutions in one or more of the following positions: 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and 274 which have proteolytic activity. In addition, a subtilisin protease is not mutated at positions Asp32, His64 and Ser221. At least one subtilisin may have SEQ ID NO:22 as described in EP 1921147, or is a variant thereof which is at least 80%, at least 90%, at least 95% or at least 98% identical SEQ ID NO:22 as described in EP 1921147 and has proteolytic activity. In one embodiment, a subtilisin is at least 80%, at least 90%, at least 95% or at least 98% identical to SEQ ID NO:22 as described in EP 1921147 and is characterized by having amino acid glutamic acid (E), or aspartic acid (D), or asparagine (N), or glutamine (Q), or alanine (A), or glycine (G), or serine (S) at position 101 (according to BPN′ numbering) and has proteolytic activity. In one embodiment, subtilisin is at least 80%, at least 90%, at least 95% or at least 98% identical to SEQ ID NO:22 as described in EP 1921147 and is characterized by having amino acid glutamic acid (E), or aspartic acid (D), at position 101 (according to BPN′ numbering) and has proteolytic activity. Such a subtilisin variant may comprise an amino acid substitution at position 101, such as R101E or R101D, alone or in combination with one or more substitutions at positions 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and/or 274 (according to BPN′ numbering) and has proteolytic activity. In one embodiment, said protease comprises one or more further substitutions: (a) threonine at position 3 (3T), (b) isoleucine at position 4 (4I), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) isoleucine at position 205 (205I), (h) aspartic acid, glutamic acid or glycine at position 217 (217D, 217E or 217G), (i) combinations of two or more amino acids according to (a) to (h). A suitable subtilisin may be at least 80% identical to SEQ ID NO:22 as described in EP 1921147 and is characterized by comprising one amino acid (according to (a)-(h)) or combinations according to (i) together with the amino acid 101E, 101D, 101N, 101Q, 101A, 101G, or 101S (according to BPN′ numbering) and has proteolytic activity. In one embodiment, a subtilisin is at least 80% identical to SEQ ID NO:22 as described in EP 1921147 and is characterized by comprising the mutation (according to BPN′ numbering) R101E, or S3T+V4I+V205I, or S3T+V4I+R101E+V205I or S3T+V4I+V199M+V205I+L217D, and has proteolytic activity. If secretion of these proteases into the fermentation medium is desired the use of the signal peptide of the AprE protein from Bacillus subtilis is preferred. In another embodiment, the subtilisin comprises an amino acid sequence having at least 80% identity to SEQ ID NO:22 as described in EP 1921147 and being further characterized by comprising S3T+V4I+S9R+A15T+V68A+D99S+R101S+A103S+I104V+N218D (according to the BPN′ numbering) and has proteolytic activity. A subtilisin may have an amino acid sequence being at least 80% identical to SEQ ID NO:22 as described in EP 1921147 and being further characterized by comprising R101E, and one or more substitutions selected from the group consisting of S156D, L262E, Q137H, S3T, R45E,D,Q, P55N, T58W,Y,L, Q59D,M,N,T, G61 D,R, S87E, G97S, A98D,E,R, S106A,W, N117E, H120V,D,K,N, S125M, P129D, E136Q, S144W, S161T, S163A,G, Y171 L, A172S, N185Q, V199M, Y209W, M222Q, N238H, V244T, N261T,D and L262N,Q,D (as described in WO 2016/096711 and according to the BPN′ numbering), and has proteolytic activity. Proteases according to the invention have proteolytic activity. The methods for determining proteolytic activity are well-known in the literature (see e.g. Gupta et al. (2002), Appl. Microbiol. Biotechnol. 60: 381-395). Proteolytic activity may be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of yellow color of free pNA which can be quantified by measuring OD405.

Preferably, the protein of interest is expressed in an amount of at least 3 g protein (dry matter)/kg fermentation broth, preferably in an amount of at least 5 g protein (dry matter)/kg fermentation broth, preferably in an amount of at least 10 g protein (dry matter)/kg fermentation broth, preferably in an amount of at least 15 g protein (dry matter)/kg fermentation broth, preferably in an amount of at least 20 g protein (dry matter)/kg fermentation broth. As the fermentation process of the present invention is suitable to provide high titers of the protein of interest, in one embodiment, the present invention refers to a method for increasing the titer of a protein of interest in a production process comprising the use of the fermentation process as described herein. Preferably, the fermentation process provides a titer of at least 5 g/l of protein of interest. More preferably, the fermentation process provides a titer of at least 10 g/l of protein of interest. Even more preferably, the fermentation process provides a titer of at least 15 g/l of protein of interest.

The compound of interest, preferably the protein of interest, may be purified from the fermentation broth by methods known in the art.

The desired compound, preferably the desired protein, may be secreted (into the liquid fraction of the fermentation broth) or may not be secreted from the host cells (and therefore is comprised in the cells of the fermentation broth). Depending on this, the desired protein may be recovered from the liquid fraction of the fermentation broth or from cell lysates. Preferably, the compound of interest is secreted by the host cell into the fermentation broth. Secretion of the compound of interest into the fermentation medium allows for a facilitated separation of the compound of interest from the fermentation medium. Recovery of the desired protein can be achieved by methods known to those skilled in the art. Suitable methods for recovery of proteins from fermentation broth include but are not limited to collection, centrifugation, filtration, extraction, and precipitation. If the protein of interest precipitates or crystallizes in the fermentation broth or binds at least in part to the particulate matter of the fermentation broth additional treatment steps might be needed to release the protein of interest from the biomass or to solubilize protein of interest crystals and precipitates. U.S. Pat. No. 6,316,240B1 and WO2008110498A1 describe a method for recovering a protein of interest, which precipitates and/or crystallizes during fermentation, from the fermentation broth. In case the desired protein is comprised in the cells of the fermentation broth release of the protein of interest from the cells might be needed. Release from the cells can be achieved for instance, but not being limited thereto, by cell lysis with techniques well known to the skilled person, e.g., lysozyme treatment, ultrasonic treatment, French press or combinations thereof.

The isolated polypeptide may then be further purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation), or extraction (see, e.g., Protein Purification, J.-C. Janson and Lars Ryden, editors, VCH Publishers, New York, 1989). The purified polypeptide may then be concentrated by procedures known in the art including, but not limited to, ultrafiltration and evaporation, in particular, thin film evaporation.

The purified protein solution may be further processed to form an “protein formulation”. “Protein formulation” means any non-complex formulation comprising a small number of ingredients, wherein the ingredients serve the purpose of stabilizing the proteins comprised in the proteins formulation and/or the stabilization of the proteins formulation itself. The term “proteins stability” relates to the retention of proteins activity as a function of time during storage or operation. The term “proteins formulation stability” relates to the maintenance of physical appearance of the proteins formulation during storage or operation as well as the avoidance of microbial contamination during storage or operation.

A “protein formulation” is a composition which is meant to be formulated into a complex formulation which itself may be determined for final use. A “protein formulation” according to the invention is not a complex formulation comprising several components, wherein the components are formulated into the complex formulation to exert each individually a specific action in a final application. A complex formulation may be without being limited thereto a detergent formulation, wherein individual detergent components are formulated in amounts effective in the washing performance of the detergent formulation. The protein formulation can be either solid or liquid. Protein formulations can be obtained by using techniques known in the art. For instance, without being limited thereto, solid enzyme formulations can be obtained by extrusion or granulation. Suitable extrusion and granulation techniques are known in the art and are described for instance in WO9419444A1 and WO9743482A1. “Liquid” in the context of enzyme formulation is related to the physical appearance at 20° C. and 101.3 kPa. Liquid protein formulations may comprise amounts of enzyme in the range of 0.1% to 40% by weight, or 0.5% to 30% by weight, or 1% to 25% by weight, or 3% to 10%, all relative to the total weight of the enzyme formulation.

The liquid protein formulation may comprise more than one type of protein. Aqueous protein formulations of the invention may comprise water in amounts of more than about 50% by weight, more than about 60% by weight, more than about 70% by weight, or more than about 80% by weight, all relative to the total weight of the protein formulation. Liquid protein formulations of the invention may comprise residual components such as salts originating from the fermentation medium, cell debris originating from the production host cells, metabolites produced by the production host cells during fermentation. In one embodiment, residual components may be comprised in liquid enzyme formulations in amounts less than 30% by weight, less than 20% by weight less, than 10% by weight, or less than 5% by weight, all relative to the total weight of the aqueous protein formulation. In one embodiment, the protein formulation, in particular the liquid protein formulation, comprises in addition to the one or more protein one or more additional compounds selected from the group consisting of solvent, salt, pH regulator, preservative, stabilizer, enzyme inhibitors, chelators, and thickening agent. The preservative in a liquid protein formulation maybe a sorbitol, a benzoate, a proxel, or any combination therefore. The stabilizers in a liquid protein formulation maybe an MPG, a glycerol, an acetate, or any combination thereof. The chelators in a liquid protein formulation maybe a citrate. Enzyme inhibitors, in particular for proteases, may be boric acid, boronic acid derivatives, in particular phenyl boronic acid derivatives like 4FPBA, or peptide aldehydes.

The protein as produced by the method of the present invention may be used in food, for example the protein can be an additive for baking. The protein can be used in feed, for example the protein is an animal feed additive. The protein can be used in the starch processing industry, for example amylases are used in the conversion of starch to ethanol or sugars (high fructose corn syrup) and other byproducts such as oil, dry distiller's grains, etc. The protein maybe used in pulp and paper processing, for example, the protein can be used for improving paper strength. In one embodiment, the protein produced by the methods of the present invention are used in detergent formulations or cleaning formulations. “Detergent formulation” or “cleaning formulation” means compositions designated for cleaning soiled material. Cleaning includes laundering and hard surface cleaning. Soiled material according to the invention includes textiles and/or hard surfaces.

The invention is further illustrated in the following examples which are not intended to be in any way limiting to the scope of the invention as claimed.

### EXAMPLES

### Example 1

Comparison of Fermentation Media with Different pH Values During Temperature Treatment Regarding Color Formation.

Cultivation Medium Composition, Treatment, and Measurement

The cultivation medium was composed with 44.5 g/L complex plant protein, NH4H2PO4, citric acid, MgSO4*7 H2O, Ca(NO3)2*4 H2O, trace element solution. Within the trace element solution is composed of (NH4)2Fe(SO4)2*6H2O, CuSO4*5H2O, ZnSO4*7H2O, MnSO4*H2O, Co(NO3)2, NiSO4*6H2O and Na2MoO4*2H2O and equimolar citric acid. Medium aliquots were adjusted with H2SO4 and NH4OH to pH 4, 5.5, 6.5 and 7.8 each. Samples were centrifuged for 10 minutes at 8500 rpm (10,000 g) and sterile filtered. L* and b* values were measured from supernatants for characterization of color development. All medium samples with pH adjustments between 4 and 7.8 were treated for 60 min at 121° C., 1 bar over pressure. Afterwards the color numbers of the supernatants were determined.

For color measurements, the samples were diluted 1:2 with deionized water in micro-cuvettes and measured by the Hach Lange LICO 690 instrument with the software CIE-Lab.

The L* value describes the color range between black and white whereby a value of 0 characterizes the color black and a value of +100 characterizes the color white.

The b* value describes the color range between blue and yellow whereby a value of −100 characterizes the color blue and a value of +150 characterizes the color yellow.

Results:

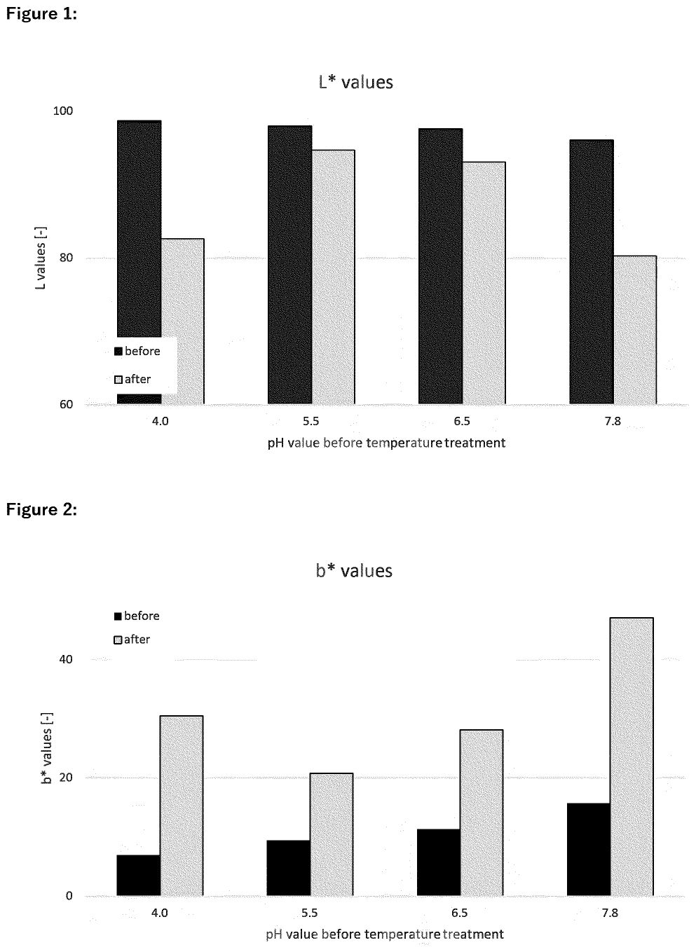

The development of the L* value is shown in FIG. 1. After temperature treatment, the L* values of all samples were lower than before temperature treatment. Thus, the fermentation medium appeared darker after temperature treatment. This effect was less pronounced when the pH of the fermentation medium was adjusted prior temperature treatment to a lower pH, i.e., pH 4.0, pH 5.5, or pH 6.5, compared to pH 7.8. This means that a low pH of the fermentation medium positively influences the color formation in the fermentation medium during temperature treatment. Thus, a low pH value prevents the formation of dark color in the fermentation media.

The development of the b* value is shown in FIG. 2. After temperature treatment the b* values of the fermentation medium were higher than before temperature treatment. Thus, after temperature treatment the fermentation medium appeared more yellow-brownish. This effect was less pronounced in fermentation media with lower pH values, i.e., pH 4.0, pH 5.5, or pH 6.5, compared to pH 7.8. Thus, a low pH value reduces the development of a yellow-brownish color.

### Example 2

Measurement of Absorption and Viscosity of Culture Media

The cultivation medium was composed with 112.5 g/L complex plant protein, K₂HPO₄, KH₂PO₄, CaCl₂)*2H₂O, MgSO₄*7H₂O, MnSO₄*H₂O, citric acid, ferric ammonium citrate and ZnSO₄*7H₂O. The fermentation medium was adjusted to pH of 4.0, 5.0, 6.0, 7.0 and 8.0 with sodium hydroxide or phosphoric acid before being autoclaved for 60 minutes at 121° C.

Autoclaved samples were spun down at 13,000 RPM in a micro centrifuge to remove solids. Thereafter, the supernatant was collected and placed in a spectrophotometer to collect absorbance readings at various wavelengths. The results are presented in Table 1.

TABLE 1
Adsorption at different wavelength at
different sterilization pH values

| | pH | | | | |
|---|---|---|---|---|---|
| Wavelength | pH 4.0 | pH 5.0 | pH 6.0 | pH 7.0 | pH 8.0 |
| 400 | 0.389 | 0.327 | 0.698 | 1.453 | 1.460 |
| 420 | 0.250 | 0.183 | 0.429 | 1.270 | 1.394 |
| 440 | 0.190 | 0.126 | 0.305 | 1.066 | 1.230 |
| 460 | 0.155 | 0.098 | 0.245 | 0.926 | 1.095 |
| 480 | 0.131 | 0.080 | 0.207 | 0.806 | 0.962 |
| 500 | 0.111 | 0.066 | 0.180 | 0.716 | 0.853 |
| 520 | 0.098 | 0.057 | 0.163 | 0.655 | 0.768 |
| 540 | 0.087 | 0.052 | 0.153 | 0.607 | 0.700 |
| 560 | 0.079 | 0.048 | 0.142 | 0.558 | 0.634 |
| 580 | 0.072 | 0.044 | 0.132 | 0.514 | 0.569 |
| 600 | 0.066 | 0.041 | 0.122 | 0.470 | 0.509 |
| 620 | 0.061 | 0.037 | 0.110 | 0.425 | 0.452 |
| 640 | 0.056 | 0.034 | 0.098 | 0.383 | 0.397 |
| 660 | 0.051 | 0.031 | 0.087 | 0.348 | 0.359 |
| 680 | 0.047 | 0.028 | 0.078 | 0.317 | 0.319 |
| 700 | 0.043 | 0.024 | 0.066 | 0.286 | 0.279 |

Table 1 shows that that at lower pH of the fermentation medium there is less absorbance at various wavelength. Hence, lowering the pH of the fermentation medium lowers color formation during temperature treatment.

Autoclaved samples were measured for viscosity at ambient temperature at a shear rate of 1000 1/s on a M3600 Grace Instrument viscosity meter. The viscosity obtained is shown in Table 2.

TABLE 2
Broth viscosity of media sterilized at different pHs.

| | pH 4.0 | pH 5.0 | pH 6.0 | pH 7.0 | pH 8.0 |
|---|---|---|---|---|---|
| Viscosity | 5.35 | 5.52 | 7.37 | 20.70 | 37.63 |
| (cP) | | | | | |

Table 2 shows that lowering the pH of the fermentation medium prior to temperature treatment reduces the development of viscosity during temperature treatment.

When samples at pH 4.0 were pH adjusted after heat treatment with sodium hydroxide to 8.0, the viscosity measurement was 5.43 cP. This demonstrates that the viscosity is not reversible, i.e., the reduced viscosity is due to the acidic pH during heat treatment and the viscosity of the culture medium itself is not pH dependent.

### Example 3

Viscosity after Fermentation

Cultivation Medium Composition and Treatment and Measurement

The cultivation medium was composed with 112.5 g/L complex plant protein, K₂HPO₄, KH₂PO₄, CaCl₂)*2H₂O, MgSO₄*7H₂O, MnSO₄*H₂O, citric acid, ferric ammonium citrate and ZnSO₄*7H₂O. The fermentation medium was adjusted to 5.0, 6.5 or 8.0 before steam in place (SIP) sterilization at 121-125° C. with constant 600 RPM agitation for 60 minutes at temperature plateau. Thereafter, the fermentation medium was adjusted to fermentation pH of 7.4.

Afterwards, the fermentation dissolved oxygen tension (DOT) was calibrated to be 100% at 10 psig and 0% at saturated nitrogen atmosphere. After dissolved oxygen probes are calibrated, inoculation ensues. Bacillus licheniformis producing a subtilisin protease was inoculated with 2% starting volume from a shake flask. Seed culture was conducted at 37° C. and harvested at exponential phase after optical density measured at a 575 nanometer wavelength reached 6 to 10 absorbance units.

The fermentation process was conducted in a baffled stirred tank reactor (STR) with pH, dissolved oxygen, and temperature probes in a medium with glucose and complex plant protein as main carbon sources. The concentration of N₂, O₂ and CO₂ were monitored by a mass spectrophotometer. For cultivations, the fed-batch mode was chosen with a starting volume of 20 L in stainless steel bioreactors with 3 Rushton turbines with 6 blades. The fermentation was tightly controlled at 20% DOT with agitation cascade up to 1100 RPM, after which the agitation is held constant. Thereafter, the dissolved is controlled with pressures of up to 20 psig. The pH is held at 7.4 and the aeration at 20 slpm, although in some instances it is lowered to control foaming. The initial glucose feed starts after glucose depletion. Oxygen uptake rate, carbon evolution rate and titers are monitored over the fermentation progression.

At quasi-steady states the oxygen uptake rate and the oxygen transfer rate are equal. Furthermore, it is well-documented that there is an inverse correlation between oxygen transfer rate and the apparent broth viscosity (cf. Ulbrecht, Jaromir J., and G. K. Patterson. “Gas into High Viscosity Liquids.” Mixing of Liquids by Mechanical Agitation. New York: Gordon and Breach, 1985. 230-31; Ryu, D. Y., and A. E. Humphrey. “Journal of Bioscience and Bioengineering.” Journal of Bioscience and Bioengineering 50 (1971): 424-31). Thus, an increased oxygen transfer at equal fermentation conditions (headspace pressure, agitation and airflow) can be considered as a surrogate indication for lower viscosities.

TABLE 3
The oxygen transfer rate is presented for fermentations
sterilized at different pHs. Similar values in the control
parameters that have an influence in oxygen transfer should
result in similar oxygen transfer rates; however, higher
OTR are observed in media sterilized at lower pH.

| | | | | |
|---|---|---|---|---|
| | Headspace | | | Average |
| Sterilization | pressure | Agitation | Airflow | OTR |
| conditions | [psig] | [RPM] | [slpm] | [mmol/L/h] |

Average OTR achieved at agitations ~500 RPM

| SIP pH 5.0 | 9.99 | 500.18 | 20.00 | 70.87 |
|---|---|---|---|---|
| SIP pH 6.5 | Not done | | | |
| SIP pH 8.0 | 9.98 | 501.19 | 20.00 | 59.23 |

Average of OTR achieved at agitations ~900 RPM

| SIP pH 5.0 | Not done | | | |
|---|---|---|---|---|
| SIP pH 6.5 | 14.99 | 911.52 | 20.00 | 138.54 |
| SIP pH 8.0 | 15.07 | 910.25 | 20.01 | 108.45 |

In addition to data obtained during the fermentation, viscosities of fermentation broth before inoculation were measured at a shear rate of 100 1/s in a M3600 Grace Instrument viscosity meter at ambient temperature. The preparation of these fermentation broths are the same as described above (pH adjust and then SIP). A spectrophotometer was also used to determine the color generated from the sterilization process and reported in Absorbance Units at 400 nanometers.

TABLE 4
Initial broth viscosities during fermentation.

| Sterilization | Viscosity | Absorbance |
|---|---|---|
| conditions | [cP] | [A.U.₄₀₀] |
| SIP pH 5.0 | 13.6 | 0.229 |
| SIP pH 6.0 | 6.80 | 0.384 |
| SIP pH 6.9 | 107 | 0.604 |

Table 3 and Table 4 show that lowering the pH in the fermentation media before temperature treatment not only reduces viscosity formation during temperature treatment of the fermentation medium, but also reduces viscosity formation during the course of fermentation.

1. A method of preparing a fermentation medium for cultivation of a microbial cell comprising
a) preparing a fermentation medium by mixing in water components supporting the growth of the cells, wherein the components comprise a complex nutrient source in an amount of 0.5-30% w/v of the fermentation medium;
b) adjusting the pH of the fermentation medium obtained in step a) to a pH between pH 3.0 and pH 6.0;
c) heating the fermentation medium obtained in step b) to a temperature above 90° C. for at least 2 min wherein the treatment of the fermentation medium in step c) is with saturated steam;
d) adjusting the pH of the fermentation medium obtained in step c) to a pH between pH 6.8 and pH 12.0.

2. The method of claim 1, wherein the complex nutrient source comprises a complex nitrogen source.

3. The method of claim 1, wherein the complex nutrient source is in an amount of 2-15% v/w of the fermentation medium.

4. The method of claim 1, wherein the complex nutrient source comprises a carbon source.

5. The method of claim 1, wherein a carbon source is added after step c) or after step d) to the fermentation medium.

6. The method of claim 1, wherein the pH in step b) is between pH 4.0.

7. A method for reducing viscosity formation in the fermentation broth during a fermentation process comprising
a. inoculating a fermentation medium obtained by a method of claim 1 with a microbial cell, preferably, a bacterial cell; and
b. incubating the fermentation medium comprising the microbial cell for a time and under conditions sufficient to allow growth of the microbial cell.

8.-14. (canceled)

15. The method of claim 2 wherein the complex nitrogen source is a complex plant protein.
